(19) United States
(12) Patent Application Publication
(10) Pub. No.: US 2015/0369830 A1
(43) Pub. Date: Dec. 24, 2015

(54) ARTICLES AND METHODS FOR RAPID THC DETECTION

(71) Applicant: THCer LLC, Red Lodge, MT (US)

(72) Inventor: Renee Marie Brent Crichlow, Robbinsdale, MN (US)

(21) Appl. No.: 14/764,360

(22) PCT Filed: Jan. 30, 2014
(86) PCT No.: PCT/US14/13950

§ 371 Date: Jul. 29, 2015

Related U.S. Application Data
(60) Provisional application No. 61/758,630, filed on Jan. 30, 2013.

(51) Int. Cl. G01N 33/94

(52) U.S. Cl. CPC G01N 33/948

(57) ABSTRACT

This disclosure describes articles for detecting THC in a saliva sample and methods of using the articles. Generally, the articles include a detection zone that generates a detectable signal that allows one to determine whether the sample contains Δ-9-tetrahydrocannabinol. The methods can produce a test result in rapid fashion, suitable for on-site testing.

### CROSS-REFERENCE TO RELATED APPLICATION

This application claims priority to U.S. Provisional Patent Application Ser. No. 61/758,630, filed Jan. 30, 2013, which is incorporated herein by reference.

### SUMMARY

This disclosure describes articles and methods for detecting THC in a saliva sample. In one aspect, this disclosure describes an article useful for detecting the presence of Δ-9-tetrahydrocannabinol in a sample. Generally, the article includes a substrate that has a detection zone and a fluid collection reservoir in fluid communication with the detection zone. The detection zone includes immobilized antibody that specifically binds Δ-9-tetrahydrocannabinol.

In some embodiments, the immobilized antibody can be immobilized to a porous matrix.

In some embodiments, the detection zone can further include a control capture area that has immobilized antibody that specifically binds a saliva component. In some of these embodiments, the saliva component can include amylase.

In some embodiments, the article can further include a rupturable reservoir that, when ruptured is in fluid communication with detection zone. The rupturable reservoir can include one or more reagents. In some of these embodiments, the rupturable reservoir can include one or more reagents for generating a detectable signal.

In some embodiments, the article can further include a cover that is at least partially transparent and covers at least a portion of the detection zone.

In some embodiments, at least a portion of the substrate can possess a structure of microfluidic transport architecture.

In another aspect, this disclosure describes a method for detecting Δ-9-tetrahydrocannabinol in a saliva sample. Generally, the method includes obtaining a saliva sample from a subject, contacting at least a portion of the sample with antibody that specifically binds THC, and detecting THC captured by the antibody.

In some embodiments, the sample is collected within two hours of the subject's most recent use of or exposure to cannabis.

In some embodiments, the method provides a result in no more than seven minutes.

In some embodiments, the method generates a detectable signal at a threshold THC concentration in the saliva sample of 14 ng/mL.

In another aspect, this disclosure describes an alternative article that may be useful for detecting the presence of Δ-9-tetrahydrocannabinol in a sample. Generally, the article includes a detection zone that includes an immobilized target compound, a sample entry zone, and a reagent zone in fluid communication with the detection zone and the sample entry zone. The reagent zone generally includes a ligand that includes a detectable label and that specifically binds the immobilized target compound.

In some embodiments, the target can be Δ-9-tetrahydrocannabinol.

In some embodiments, the detection zone includes the target compound in a complex with a carrier. In some of these embodiments, the carrier can include bovine serum albumin.

In some embodiments, the article can further include an absorbent reservoir in fluid communication with the detection zone and positioned opposite the reagent zone with respect to the detection zone.

In some embodiments, the detection zone can include a fluid transport membrane.

In some embodiments, at least one surface providing fluid communication between two zones can include microfluidic architecture.

In some embodiments, the article can further include a control target in the detection zone and a control ligand in the reagent zone. The control ligand can include a detectable label and specifically bind to the control target.

In yet another aspect, this disclosure describes a method for detecting Δ-9-tetrahydrocannabinol in a saliva sample. Generally, this method includes obtaining a saliva sample from a subject, contacting at least a portion of the sample with the article that generates a detectable signal, and detecting a detectable signal generated by the detectable label. Generally, the article includes a detection zone that includes an immobilized target compound, a sample entry zone, and a reagent zone in fluid communication with the detection zone and the sample entry zone. The reagent zone generally includes a ligand that includes a detectable label and that specifically binds the immobilized target compound.

In some embodiments, the sample is collected within two hours of the subject's most recent use of or exposure to cannabis.

In some embodiments, the method provides a result in no more than seven minutes.

In some embodiments, the method generates a detectable signal at a threshold THC concentration in the saliva sample of 14 ng/mL.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. A perspective view of one embodiment of a detection article.

FIG. 2. A side view of one embodiment of a detection article.

FIG. 3. (A) a perspective view of an unused detection article ready for use; (B) a perspective view of the detection article shown in (A) after performing the assay and showing a negative test result; (C) a perspective view of the detection article shown in (A) after performing the assay and showing a positive test result; (D) a perspective view of the detection article shown in (A) after performing the assay and showing an inconclusive result; (E) a perspective view of the detection article shown in (A) after performing the assay and showing an inconclusive result.

FIG. 4. A side view of one embodiment of a detection article.

FIG. 5. A perspective view of one embodiment of a detection article.

FIG. 6. (A) a perspective view of the detection article shown in FIG. 5 after performing the assay and showing a negative test result; (B) a perspective view of the detection article shown in FIG. 5 after performing the assay and showing a positive test result.

### DETAILED DESCRIPTION OF ILLUSTRATIVE EMBODIMENTS

This disclosure describes articles and methods related to human non-invasive drug testing. Detecting marijuana use through testing of oral fluid is of interest to health professionals, law enforcement, and others concerned about personnel impairment from drug intoxication. The sampling of oral fluid for marijuana use as described herein exploits the relative ease of obtaining and testing the deposition of cannabinoid compounds in the oral mucosa following smoking of the drug. Additionally, the measurement of the marijuana-specific compound Δ-9-tetrahydrocannabinol (THC) is readily testable from oral fluid using the methods and articles described herein and is of particular interest because it remains present in the oral fluid following the onset and subsequent decline in physiological and pharmacological effects of marijuana. The methods and articles described herein are specifically designed to distinguish acute ingestion within the time period of greatest impairment. Moreover, the articles and methods allows for detection of an analyte from an unprocessed saliva sample—i.e., the assay permits detection of the THC target in a non-purified form.

Conventional testing regimens for detecting and/or quantifying drug concentrations in oral fluid include gas chromatography-mass spectrometry (GC-MS) methods (typically limited to a laboratory environment) and immunoassay methods (often used for field testing). The confirmation of drugs in oral fluid has proven a challenge to toxicologists due to limited sample volumes available for analysis, the stabilization and preservation of samples until they reach the laboratory, and low cutoff concentrations in all cases. Tandem-MS can be used to achieve increased sensitivity with small sample volumes. (Niedbala, et al., 2004, J. Anal Toxicol 28:546-552). Chromatography coupled with MS is often used for confirming the presence of drugs in biological matrices. GC-MS are routinely used in analytical laboratories, as they provide definitive identification of drug chemicals of interest. Gas chromatography-mass spectrometry methods are, however, impractical, difficult to administer, and/or too costly for field testing. Specifically, for roadside sobriety examinations by law enforcement or in-office examinations by a medical provider, the window for testing is very short, typically less than 10 minutes from test administration to the need for test results. Thus, most rapid screening tests for drugs-of-abuse are immunoassays. Although less accurate and more costly on a per-test basis, antibodies are used in immunoassays for detection of drugs in saliva that must cross-react with the parent drug and lipophilic metabolites. For cannabis (marijuana), 9-tetrahydrocannabinol predominates in saliva. When drugs are leached into saliva from buccal depots such as is the case for smoked drugs, such as marijuana, parent drug and pyrolysis products will predominate in saliva.

A study conducted in 1999, however, assessed three onsite tests, Securetec Drugwipe (Affiniton, Williamsport, Pa.) the Avitar Ora|Screen, and the RapiScan and concluded that the sensitivity of the devices was insufficient for low-drug concentrations seen for cannabinoid and the average total time for sampling and analysis was considered too long for roadside/in-office use at 20 minutes. (Cozart Biosciences, Ltd., UK) (Moore et al., 2007, J Anal Toxicol. 31(4):187-194). A follow-up study, started in 2003, found that six of the nine devices evaluated had an unacceptable number of failures (greater than 25%). In addition, the sensitivity and specificity of the devices did not meet the minimum specifications and no single device could be recommended for use at the roadside (Walsh et al., 2003, J Anal Toxicol. 27(7):429-439).

Existing commercially-available saliva tests fail to distinguish between acute exposure and chronic ingestion of marijuana in an inexpensive, categorical, go/no-go rapid test suitable for roadside or medical clinic screening. Additionally, no saliva-based rapid THC test is being used for law enforcement roadside testing other than limited trials with dedicated testing equipment that is unwieldy for widespread use in the field (Walsh et al., 2003, J Anal Toxicol. 27(7):429-439). Marijuana can be present in the serum long after its intoxicating effects have waned. In a shifting terrain of law enforcement needs across the country, a test that distinguishes between legal chronic use, such as with medical marijuana, and acute ingestion would be helpful to law enforcement, employers, and employees.

The methods and articles described herein are designed and calibrated to detect THC at a predetermined threshold concentration in oral fluid, which corresponds to THC levels present in oral fluids during the period of likely impairment following acute ingestion, typically around two hours. The articles and methods described herein allow one to use oral fluids to rapidly distinguish between acute ingestion during the period of greatest impairment and chronic or more remote ingestion of THC. In some cases, the articles and methods described herein permit one to detect THC in the saliva at or above the threshold level of 14 ng/ml within seven minutes.

Thus, in one aspect, this disclosure describes articles that may be used to rapidly test saliva for the presence of Δ-9-tetrahydrocannabinol (THC). Generally, in some embodiments, the article includes a substrate that includes a detection zone and a fluid collection reservoir. The detection zone generally includes antibody that specifically binds Δ-9-tetrahydrocannabinol. Moreover, the substrate provide fluid communication between the fluid collection reservoir and the detection zone.

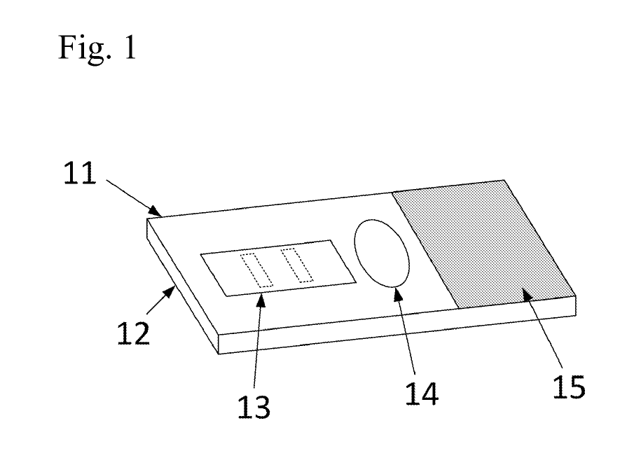

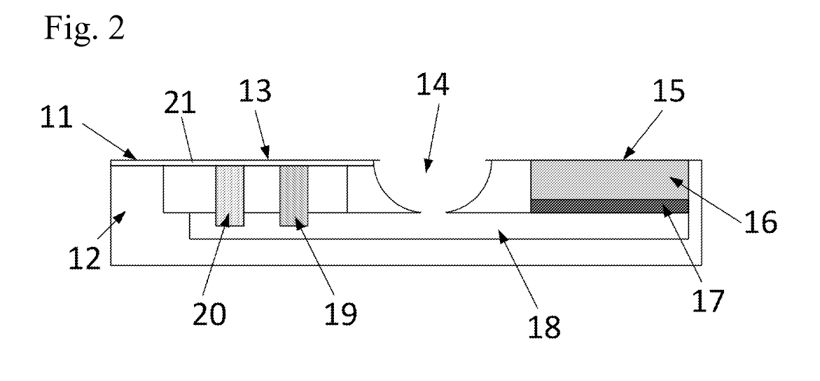

FIG. 1 illustrates one embodiment of the article (11), which includes a substrate (12), a fluid collection reservoir (14), and a detection zone (13). The substrate (12) includes a fluid collection reservoir (14) in which sample saliva is collected. The substrate (12) provides fluid communication between the fluid collection reservoir (14) and the detection zone (13), perhaps best illustrated in the side view of FIG. 2. In some embodiments, the substrate (12) can include microfluidic transport architecture to promote fluid transport from one area of the article to another—e.g., from the fluid collection reservoir (14) to the detection zone (13). As used herein, microfluidic transport architecture refers to one or more fluid transport structures arranged in a pre-determined, self-contained pattern. For example, microfluidic transport architecture can includes at least one structure having a dimension no greater than about 1000 micrometers such as, for example, a microchannel, a fluid reservoir, a sample handling region, or any combination thereof. In some embodiments, one or more reagents, described in more detail below, may be coated on the surface of the substrate (12) in an area of microfluidic transport architecture. In some cases, the microfluidic transport architecture may be integral to the substrate (12). In other embodiments, the microfluidic transport architecture may be provided in a film or other layer that is affixed, adhered, or otherwise attached to a surface of the substrate (12).

The detection zone (13) includes at least one capture area (19) that can include immobilized antibody that specifically binds Δ-9-tetrahydrocannabinol (THC)—i.e., THC capture antibody. As used herein, “specific” and variations thereof refer to having a differential or a non-general (i.e., non-specific) affinity, to any degree, for a particular target. Also as used herein, the term “antibody” generally refers to a preparation that includes at least one species of immunoglobulin or fragment thereof (e.g., scFv, Fab, F(ab′)₂, Fv, or modified forms thereof). Thus, the term “antibody” may include a polyclonal antibody preparation, a monoclonal antibody, a fragment of an immunoglobulin, or any combination thereof. Antibody that specifically binds to THC is commercially available. (e.g., Lifespan Biosciences, Inc., Seattle, Wash.; Epitomix Inc., Burlingame, Calif.).

The antibody may be immobilized to form a capture area (19). FIG. 2 illustrates the detection zone (13) as a porous matrix impregnated with immobilized antibody to form distinct capture areas (19, 20). Alternatively, the antibody may be immobilized to one or more surfaces of the channel (18). In some of these embodiments, the antibody may be immobilized in an area of microfluidic architecture.

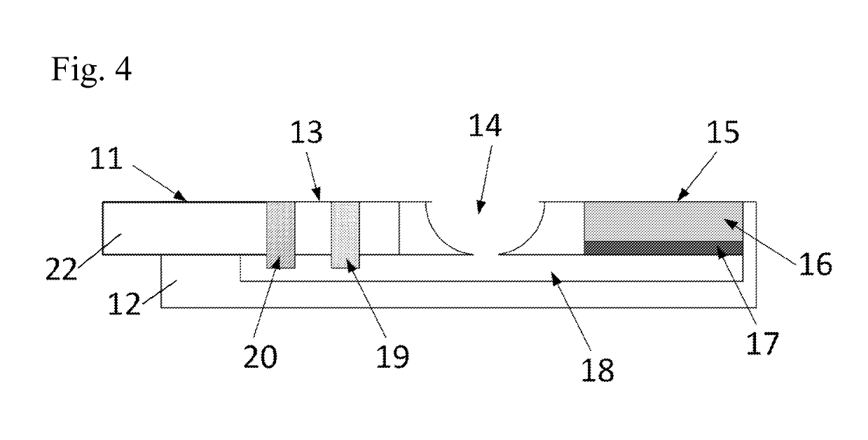

FIG. 4 illustrates an alternative embodiment in which the detection zone (13) includes a matrix configured to extend beyond the edge of the substrate (12). In use, the matrix extension (22) may be placed under the tongue of the subject being tested in order to collect a sample of saliva. In such embodiments, a sample may be collected using the fluid collection reservoir (14) and/or the matrix extension (22).

The antibody may be immobilized in the detection zone (13) in any manner that permits (a) contact between the immobilized antibody and at least a portion of the saliva sample that is subject to fluid transport from either the fluid collection reservoir (14) through channel (18) to the detection zone (13) or through the matrix extension (22) and (b) visualization of captured THC that is subsequently labeled. In embodiments in which the detection zone (13) includes a porous matrix, as shown in FIG. 2 and FIG. 4, the matrix can decrease the rate at which the sample fluid may dry.

In some embodiments, fluid transport from the fluid collection reservoir (14) to the detection zone (13) may be facilitated by a reagent solution (16) released from a rupturable fluid reservoir (15). In other embodiments, the reagent solution (16) may be added to the sample in the fluid collection reservoir (14). In some embodiments, one or more reagents may be coated or otherwise deposited on the surface of the substrate (12) in an area of microfluidic transport architecture. Prior to rupture of the membrane (17), the reagent solution (16) may be sequestered from the channel (18). When the membrane (17) is ruptured, the channel (18) provides fluid communication between the rupturable fluid reservoir (15), the sample collection reservoir (14) and the capture area (13). Construction of the article (11) to include the reagent solution (16) in the rupturable fluid reservoir (15) permits convenient dispensing of the reagent solution (16). In this embodiments, the article (11) can be constructed to contain all components—except the sample—necessary to store, release, contain, and allow to react all required reagents of the immunoassay.

The reagent solution (16) that, in some embodiments, is sequestered in the rupturable fluid reservoir (15) can include materials such as, for example, reagents and/or a carrier to facilitate fluid transport of a portion of the sample from the sample collection reservoir (14) to the detection zone (13). Other suitable materials that may be included in the reagent solution (16) include, for example, reagents for generating a detectable signal in the event that a target (e.g., Δ-9-tetrahydrocannabinol or the positive control substance) is captured in the THC capture area (19) and/or, if present, the control capture area (20). Since Δ-9-tetrahydrocannabinol is hydrophobic, the reagent solution (16) can include one or more materials selected to assist fluid transport of Δ-9-tetrahydrocannabinol into the detection zone (13) and capture area (19) such as, for example, a surfactant solution. As used herein, the reagent solution can be a true solution or may be a suspension, emulsion, or any other mixture of components in a liquid base.

One performing the assay can collect a sample, either by placing the matrix extension (22), if present, in the mouth of a subject (e.g., under the tongue) or by having the subject deposit a sample in the sample collection reservoir (14). The assay may be activated by releasing the reagent solution (16). In embodiments in which the reagent solution is sequestered in a rupturable fluid reservoir (15), one can activate the assay by applying sufficient pressure to the rupturable fluid reservoir (15) to rupture the membrane (17) and release the reagent solution (16) into the channel (18). The membrane (17) may be formed of any non-porous, rupturable material. In embodiments in which the article lacks a rupturable fluid reservoir (15), the reagent solution (16) may be released by adding an appropriate volume of the reagent solution to the article (e.g., in a dropwise manner) such as, for example, by depositing the reagent solution (16) into the fluid collection reservoir (14).

In the embodiment illustrated in FIGS. 1-4, the detection zone (13) includes a capture area (19) that includes immobilized antibody for capturing Δ-9-tetrahydrocannabinol that may be present in a sample. In some embodiments, the detection zone (13) can include a second capture area (20) that includes antibody that specifically binds to a positive control component of saliva—i.e., a component of saliva that is typically present regardless of whether the subject being tested has ingested marijuana. In some embodiments, the saliva component to which the control capture area antibodies bind can include amylase, a protein prevalent in saliva. Antibody that specifically binds to amylase is commercially available (e.g., Lifespan Bioscience, Inc. Seattle, Wash.).

At least a portion of a collected sample is transported to the detection zone (13) from its site of collection, either via the matrix extension (22) or via the sample collection reservoir (14) and through the channel (18). The portion of the sample may be allowed to be transported to the detection zone (13) prior to adding the reagent solution (16). In other embodiments, whether released from the rupturable fluid reservoir (15) or added to the sample collection reservoir (14), the reagent solution may help facilitate fluid transport of at least a portion of the sample to the detection zone (13).

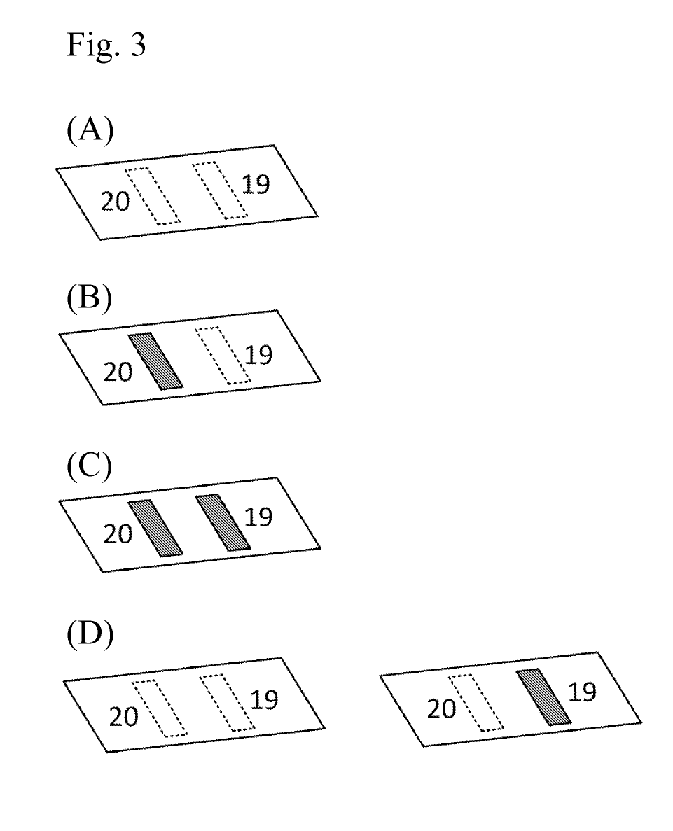

THC (Δ-9-tetrahydrocannabinol), if present in the sample, will bind to the immobilized Δ-9-tetrahydrocannabinol capture antibody in the capture area (19), where it then becomes mobilized and is subject to labeling and detection by components of the reagent solution (16). The generation of a detectable signal in capture area (19) indicates presence of THC in the saliva sample at a concentration of at least a predetermined threshold level, discussed in more detail below. When the control capture area (20) is present, the antibody immobilized in the control capture area (20) immobilizes the saliva component (e.g., amylase) to which it specifically binds, where it is subject to labeling and detection by components of the reagent solution (16). When present, the generation of a detectable signal in the control capture area (20) signifies that the assay functioned properly so that the absence of a detectable signal in the THC capture area (19) can be interpreted as a negative result. (FIG. 3B).

The detectable signal may be generated by one or more components of the reagent solution (16). In some embodiments, the reagent solution (16) can include one or more reagents necessary to generate a fluorescent or colorimetric signal. Such reagent can include a detection antibody that specifically binds to a target captured in the detection zone (13)—e.g., THC captured in the THC capture area (19) or, if a control capture area (20) is present, the saliva component (e.g., amylase) captured in the control capture area (20).

FIG. 3 illustrates the detection area (13) of an embodiment of the article that includes a THC capture area (19) and a control capture area (20) both prior to use (FIG. 3A) and after performing the assay (FIG. 3B-E). As described above, FIG. 3B shows an example of a negative result. FIG. 3C shows an example of a positive result—a detectable signal is generated in both the THC capture area (19) and the control capture area (20). FIG. 3D and FIG. 3E illustrate examples of inconclusive results—i.e., results that are neither positive nor negative, but indicate a problem with the assay. When obtaining such results, it is recommended that the test be performed again.

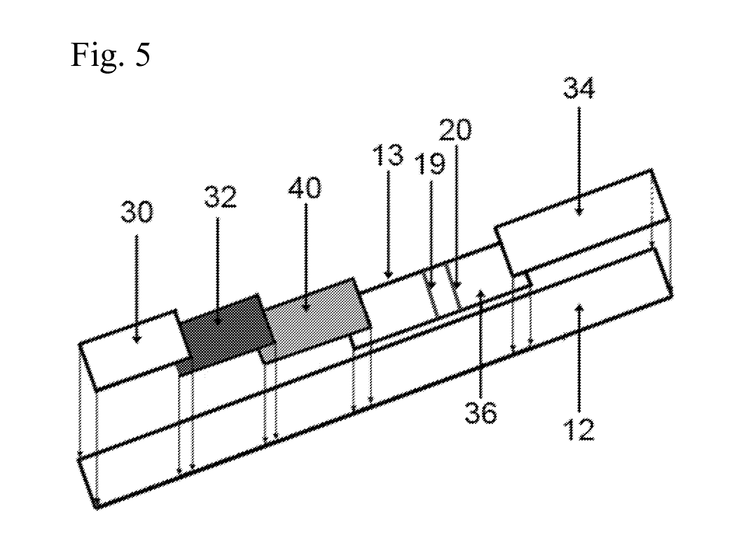

FIG. 5 illustrates an alternative embodiment designed for performing a competition assay. The details and construction of this embodiment are described in U.S. Pat. Nos. 7,344,893; 7,910,384; and/or 8,153,444. Generally, in this embodiment, the article includes a substrate (12) that includes a detection zone (13) and a sample entry zone (30) in fluid communication with the detection zone (13). The sample entry zone (13) may be manufactured of an absorbent material for absorbing a liquid sample. In some embodiments, such as those described in one or more of the patents listed immediately above, the liquid sample may be applied to a collection area of the article (not shown) and transported to the sample entry zone (13). In other embodiments, the sample entry zone (30) may be designed for directly receiving the liquid sample.

The article further includes a reagent zone (32) that includes a ligand that specifically binds to Δ-9-tetrahydrocannabinol. The ligand further includes a detectable label. For embodiments in which the detection zone (13) includes a control capture area (20), the reagent zone (32) can further include a control ligand that includes a detectable label and specifically binds to the control capture area (20) in the detection zone (13). The reagent zone (32) is provided in fluid communication between the sample entry zone (30) and the detection zone (13) so that at least a portion of the liquid sample passes through the reagent zone (32) as it is transported from the sample entry zone (30) to the detection zone (13).

The detectable label can be any label that is visible when a sufficient number of ligand molecules carrying the detectable label are captured in the detection zone (13). Exemplary detectable labels include conjugated gold particles as described in U.S. Pat. Nos. 7,344,893; 7,910,384; and/or 8,153,444, a colorimetric label, a fluorescent label, etc.

The embodiment shown in FIG. 5 includes a detection zone (13) that includes a THC capture area (19) and a control capture area (20). As shown in FIG. 5, the detection zone (13) is located on a portion of a fluid transport membrane (36) that is disposed on at least a portion of the substrate (12). In alternative embodiments, fluid transport through the detection zone may be accomplished without the use of a fluid transport membrane (36), but rather by integrating microfluidic architecture, as described in more detail below, directly into the substrate (12).

The THC capture area (19) includes a target immobilized to at least a portion of the detection zone, either the substrate (12) or, if present, the fluid transport membrane (36). The target may be Δ-9-tetrahydrocannabinol or an analog of Δ-9-tetrahydrocannabinol that binds to the ligand provided in reagent zone (32). The target may be directly immobilized to the substrate (12) or, if present, the fluid transport membrane (36) or may be complexed with a carrier (e.g., bovine serum albumin, BSA). When complexed with a carrier, the complex may be immobilized to the substrate (12) or, if present, the fluid transport membrane (36) in any manner that permits the target to be accessible for binding by components of the liquid sample as at least a portion of the liquid sample passes through the detection zone (13).

In some embodiments, the substrate (12) and/or the fluid transport membrane (36), if present, can include microfluidic transport architecture to promote fluid transport from one area of the article to another—e.g., from the sample entry zone (30) to the detection zone (13). In some cases, the microfluidic transport architecture may be integral to the substrate (12). In other embodiments, the microfluidic transport architecture may be provided in a film or other layer that—e.g., the fluid transport membrane (36)—is affixed, adhered, or otherwise attached to a surface of the substrate (12).

In other embodiments, the article can include an absorbent reservoir (34) in fluid communication with the detection zone (13) opposite the sample entry zone (30). When present, the absorbent reservoir (34) can promote fluid transport by drawing fluid from the sample entry zone (30) and through the detection zone (13). The absorbent reservoir can be constructed of any suitable absorbent material. Suitable absorbent materials include fibrous textile type materials, including woven, non-woven, knit, and stitch bonded materials or absorbent foams. Alternatively, the absorbent can include an absorbent polymer such as a hydrocolloid or hydrophilic polymer such as a supersorber. A hydrocolloid (e.g., starch, modified cellulose, gelatin or other protein, polysaccharide, etc.) or supersorber (e.g., modified starch, acrylates, starch/acrylate copolymers, acrylamides, other vinyl polymers, etc.) may be immobilized in a matrix such as a hydrophobic matrix of conventional hydrocolloid dressings or may alternatively be part of a hydrophilic gel matrix (e.g., a UV or E-beam cured acrylate). Alternatively, the absorbent may include both a fibrous textile and an absorbent polymer.

The substrate in embodiment illustrated in FIG. 1 or the embodiment illustrated in FIG. 4 may be manufactured of any suitable material. In many cases, the substrate may be formed from a material sufficient rigid that the substrate can maintain its shape during the testing process and/or resist breakage. Suitable materials include, for example, polymeric materials such as, for example, thermoplastic materials such as polyolefins, polyesters, polyamides, poly(vinyl chloride), polyether esters, polyimides, polyesteramide, polyacrylates, polyvinylacetate, hydrolyzed derivatives of polyvinylacetate, etc., or combinations thereof. In some embodiments, the substrate may be formed from a polyolefin, particularly polyethylene, polypropylene, a blend and/or a copolymer thereof, or a copolymer of propylene and/or ethylene with minor proportions of other monomers, such as vinyl acetate or acrylates such as methyl and butylacrylate. Polyolefins can confer desirable physical properties to the substrate, are typically easy to process, and are typically lower cost than other thermoplastic materials having similar characteristics. Polyolefins also can readily replicate the surface of a casting or embossing roll. They are tough, durable, and hold their shape well, thus making such films easy to handle after manufacture. In other embodiments, the substrate may be formed from a hydrophilic polyurethanes. Alternatively, the substrate can be cast from a thermosets (curable resin materials) such as, for example, a polyurethane, an acrylate, an epoxy or a silicone, and cured by exposure to heat, light, UV radiation, E-beam radiation, or moisture. Exemplary suitable materials for substrate include poly(methylmethacrylate) polycarbonates, polyesters, and polyimides. Examples of suitable photocurable resin compositions include, for example, alkyl acrylates and methacrylates (e.g., polymethyl methacrylate). The substrate may contain various additives including, for example, a surface energy modifier (e.g., a surfactant and/or hydrophilic polymer), a plasticizer, an antioxidant, a pigment, a release agent, an antistatic agent, and/or a treatment to render the film biocompatible (e.g., a heparin coating).

In some embodiments, the detection zone (13) may be enclosed by an at least partially transparent cover (21) to maintain the integrity of the detection zone and capture area (19 and, when present, 20) before, during, and/or after performing the assay. The cover (21) is illustrated in FIG. 2 in the context of one embodiment of the article. The embodiment of the article illustrated in FIG. 5 may similarly include a cover (21). The cover (21) may be formed from any suitable material that provides sufficient transparency to permit one to visualize the results of the assay in the detection zone (13). Accordingly, the cover (21) need not provide absolute transparency. Suitable materials from which the cover (21) may be formed include, for example, a polymeric material or glass. Polymeric materials often provide sufficient transparency and desirable flexibility and/or durability compared to glass.

At least a portion of a collected sample is introduced into the sample entry zone (30), either directly or, as described above, via transport from a sample collection area. At least a portion of the collected sample is transported to the detection zone (13) from the sample entry zone (30). As a portion of the sample passes through the reagent zone (32) where it will mix with the ligand that specifically binds to Δ-9-tetrahydrocannabinol and, if present, the control ligand.

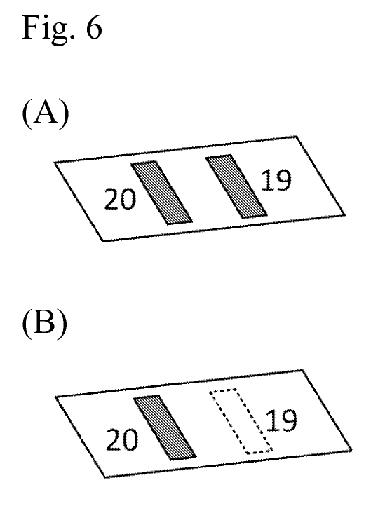

In a sample with no THC, the ligand will mix with the sample, but will remain unbound to any component of the sample. The ligand will therefore be available to bind to the target in the THC capture area (19) generating a detectable signal and a negative test result. (FIG. 6A).

In a sample that contains THC, the ligand will mix with the sample and will bind to THC in the sample. The ligand with therefore be occupied and will fail to bind to the target in the THC capture area (19). Consequently, no detectable signal will be generated, producing a positive test result. (FIG. 6B).

When the article includes a control capture area (20), the reagent zone (32) can include a ligand that binds to a control target provided in the control capture area (20). In one embodiment, the control capture target can include, for example, biotin conjugated to a protein that is immobilized to the substrate (12) or, if present, the fluid transport membrane (36). In such an embodiment, the control ligand can include streptavidin that is conjugated or otherwise attached to a detectable label. The control ligand will bind to the control target as fluid passes through the control capture area (20). The labeled control ligand will therefore produce a detectable signal (FIGS. 6A and 6B), indicating that the assay is functioning properly. If the control capture area (20) is present and control ligand is provided in the reagent zone (32), but a detectable signal does not appear in the control capture area, the results of the assay are inconclusive—i.e., results that are neither positive nor negative, but indicate a problem with the assay. When obtaining such results, it is recommended that the test be performed again.

The articles and methods described above can provide rapid immunodetection of THC in saliva. Moreover, the articles and methods may be designed to distinguish between acute exposure and chronic ingestion of marijuana. As used herein, “acute” exposure refers to exposure at a level commonly associated with physical and/or neurological impairment; as used herein, “chronic” ingestion refers to ingestion of such low exposure or with sufficient time lapse so that the subject exhibits no significant level of physical or neurological impairment.

In some embodiments, acute exposure can include use of or other exposure to cannabis with no more than three hours such as, for example, no more than 150 minutes, no more than 120 minutes, no more than 100 minutes, no more than 90 minutes, no more than 80 minutes, no more than 70 minutes, no more than 60 minutes, no more than 50 minutes, no more than 40 minutes, or no more than 30 minutes. In some embodiments, acute exposure can include use of or other exposure to cannabis within two hours.

In addition, the article can be calibrated to generate a detectable signal when a tested sample possesses a predetermined threshold level of THC that reflects acute exposure, but to not generate a detectable signal when a tested sample reflects chronic ingestion. In this way, the articles and methods described herein can assist law enforcement, medical professionals, employers, employees, parents, etc. determine whether a tested sample reflects cannabis use that can impair the function of the subject. In some embodiments, the predetermined threshold level of THC in the saliva can be at least 14.0 ng/mL such as, for example, at least, 14.2 ng/mL, 14.4 ng/mL, 14.6 ng/mL, 14.8 ng/mL, 15.0 ng/mL, 15.2 ng/mL, 15.4 ng/mL, 15.6 ng/mL, 15.8 ng/mL, or at least 16.0 ng/mL.

The assay may be performed on a sample having any suitable volume. In some embodiments, the sample may have a minimum volume of at least 1 μl such as, for example, at least 5 μl, at least 10 μl, at least 15 μl, at least 25 μl, at least 50 μl, at least 75 μl, at least 100 μl, at least 125 μl, or at least 150 μl. The maximum sample volume can be any volume capable of being contained by the device and is, therefore, more a function of preferred design parameters than any technical limitation. In some embodiments, therefore, the sample may have a maximum volume of no more than 5 ml such as, for example, no more than 2 ml, no more than 1 ml, no more than 500 μl, no more than 250 μl, no more than 200 μl, no more than 150 μl, no more than 100 μl, no more than 75 μl, or no more than 50 μl. In some embodiments, the sample volume may be expressed as a range having endpoints defined by any minimum volume listed above and any maximum volume listed above that is greater than the minimum volume.

This is a novel application of immunoassay for a rapid non-invasive testing article using oral fluid. The articles described herein and the methods employing those articles provide sensitivity and specificity consistent with a goal of screening for THC acute ingestion. A two-hour time frame, for example, can correlate with a THC level of at least 14 ng/mL in saliva. The acute exposure detection period and threshold level is unique to THC detection in saliva and allows rapid discrimination between acute ingestion during the period of greatest impairment and, on the other hand, chronic ingestion of THC lingering in the test subject. The article may be calibrated so that a detectable signal is generated when a tested sample contains the predetermined threshold level of THC. In some embodiments, the calibration can involve controlling the density of THC capture antibody in the THC capture area (19). In other embodiments, the calibration can involve controlling the concentration of the a ligand that specifically binds to Δ-9-tetrahydrocannabinol provided in the reagent zone (32).

The detection method described herein can provide a test result in no more than 20 minutes such as, for example, no more than 15 minutes, no more than 14 minutes, no more than 13, minutes, no more than 12, minutes, no more than 11 minutes, no more than 10 minutes, no more than nine minutes, no more than eight minutes, no more than seven minutes, no more than six minutes, or no more than five minutes.

Various embodiments of the articles and methods described herein address certain challenges not addressed by conventional THC detection assays. First, saliva is composed of 99% water, 0.7% protein (largely amylase), and 0.26% mucins. In healthy donors, gingival crevicular fluid from the tooth/gum margin may constitute up to 0.5% of oral fluid collected and is of similar composition to that of plasma. Saliva is an acceptable and well-documented medium for drug screening. However, a collection apparatus that non-invasively obtains saliva and delivers it to a testing zone within the rapid test device is specific to the articles described herein and the use of those articles.

Second, cannabinoids are challenging to detect in oral fluid. The detectability of THC is severely limited by its poor solubility in water (only 2.8). However, the solubility of THC improves in the blood and saliva, allowing concentrations of <1 ng/mL to be detected by some analyses. The articles and methods described herein use a novel immunoassay testing apparatus to rapidly test concentrations of THC.

Third, most drugs enter saliva by passive diffusion across the cell membranes. As a result, there are time delay and/or half-life concentration concerns with obtaining accurate measurements of cannabinoids in saliva. Saliva concentrations of drugs that cross the cells by passive diffusion are related to blood and/or plasma concentrations of the unbound, unionized parent drug and/or its lipophilic metabolites. The theoretical saliva:plasma ratio (S/P ratio) can be calculated from an equation derived from the Henderson-Hasselbach equation (acidic drugs):

pH=pKₐ+log([A⁻]/[HA])

where in the derived equation, S/P is the saliva to plasma ratio, S is the drug concentration in saliva and P is the drug concentration in plasma, pK_{b} is the log of the ionization constant for basic drugs, pKₐ is the log ionization constant for acidic drugs, pHₛ is the pH of saliva, pHₚ is the plasma pH, fₚ is the fraction of drug protein bound in plasma, and fₛ is the fraction protein bound in saliva. This equation can be used to extrapolate the plasma content of THC from oral fluid, and thus determine an intoxication level of the test subject. The articles and methods described in this disclosure use this degradation fundamental to better estimate THC concentrations for the testing regimen and results display.

Fourth, acidic drugs with a pKₐ less than 5.5 (e.g., cannabinoids) and drugs that are highly protein bound generally have a S/P ratio of less than 1.0. This complicates the robustness of any oral fluid field test for drug compounds, but is addressed by the articles and methods described in this disclosure.

Finally, drugs-of-abuse can be classified into those that enter oral fluid by passive diffusion and those that enter oral fluid from depots in mouth tissues. In general, drugs that enter the oral fluid from depots in the oral tissues are found in higher concentrations than expected from their theoretical S/P ratios calculated from the Henderson-Hasselbach equation. Drugs that are abused are often smoked. Smoking cannabis can create oral tissue depots having S/P ratios that can be elevated more than a 100-fold. The articles and methods described herein exploit this relationship for obtaining satisfactory results rapidly.

### EXEMPLARY EMBODIMENTS

### Embodiment 1

An article comprising a substrate comprising a detection zone comprising immobilized antibody that specifically binds Δ-9-tetrahydrocannabinol and a fluid collection reservoir in fluid communication with the detection zone.

### Embodiment 2

The article of Embodiment 1 wherein the immobilized antibody is immobilized to a porous matrix.

### Embodiment 3

The article of Embodiment 1 or Embodiment 2 wherein the detection zone further comprises a control capture area that comprises immobilized antibody that specifically binds a saliva component.

### Embodiment 4

The article of Embodiment 3 wherein the saliva component comprises amylase.

### Embodiment 5

The article of any preceding Embodiment wherein the substrate further comprises a rupturable reservoir comprising a regent solution, wherein when the reservoir is ruptured, the reagent solution is in fluid communication with detection zone.

### Embodiment 6

The article of Embodiment 5 wherein the reagent solution comprises a reagent for generating a detectable signal.

### Embodiment 7

The article of Embodiment 5 wherein the reagent solution comprises a surfactant solution.

### Embodiment 8

The article of any preceding Embodiment further comprising a cover that is at least partially transparent and covers at least a portion of the detection zone.

### Embodiment 9

The article of any preceding Embodiment wherein the substrate comprises microfluidic transport architecture.

### Embodiment 10

A method for detecting Δ-9-tetrahydrocannabinol in a saliva sample, the method comprising:

obtaining a saliva sample from a subject;

contacting at least a portion of the sample with antibody that specifically binds THC; and

detecting THC captured by the antibody.

### Embodiment 11

The method of Embodiment 10 wherein the sample is collected within two hours of the subject's most recent use of or exposure to cannabis.

### Embodiment 12

The method of Embodiment 10 or Embodiment 11 wherein the method provides a result in no more than seven minutes.

### Embodiment 13

The method of any one of Embodiments 10-12 wherein the method generates a detectable signal at a threshold THC concentration in the saliva sample of 14 ng/mL.

### Embodiment 14

An article comprising:

a detection zone comprising an immobilized target compound;

a sample entry zone; and

a reagent zone in fluid communication with the detection zone and the sample entry zone, wherein the reagent zone comprises a ligand that comprises:
specific binding affinity for the immobilized target compound; and
a detectable label.

### Embodiment 15

The article of Embodiment 14 wherein the target comprises Δ-9-tetrahydrocannabinol.

### Embodiment 16

The article of Embodiment 14 or Embodiment 15 wherein the detection zone comprises the target compound in a complex with a carrier.

### Embodiment 17

The article of Embodiment 16 wherein the carrier comprises bovine serum albumin.

### Embodiment 18

The article of any one of Embodiments 14-17 further comprising an absorbent reservoir in fluid communication with the detection zone and positioned opposite the reagent zone with respect to the detection zone.

### Embodiment 19

The article of any one of Embodiments 14-18 wherein the detection zone comprises a fluid transport membrane.

### Embodiment 20

The article of any one of Embodiments 14-19 wherein at least one surface providing fluid communication between two zones comprises microfluidic architecture.

### Embodiment 21

The article of any one of Embodiments 14-20 wherein:

the detection zone further comprises a control target; and

the reagent zone comprises a control ligand that comprises:
specific binding affinity for the control target; and
a detectable label.

### Embodiment 22

A method for detecting Δ-9-tetrahydrocannabinol in a saliva sample, the method comprising:

obtaining a saliva sample from a subject;

contacting at least a portion of the sample with the article of any one of Embodiments 14-21; and

detecting a detectable signal generated by the detectable label.

### Embodiment 23

The method of Embodiment 22 wherein the sample is collected within two hours of the subject's most recent use of or exposure to cannabis.

### Embodiment 24

The method of Embodiment 22 or Embodiment 23 wherein the method provides a result in no more than seven minutes.

### Embodiment 25

The method of any one of Embodiments 22-24 wherein the method generates a detectable signal at a threshold THC concentration in the saliva sample of 14 ng/mL.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Unless otherwise specified, the particular examples, materials, amounts, and procedures described herein are exemplary and are to be interpreted broadly in accordance with the scope and spirit of the invention as set forth herein.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

The complete disclosure of all patents, patent applications, and publications cited herein are incorporated by reference in their entirety. In the event that any inconsistency exists between this disclosure of this application and the disclosure(s) of any document incorporated herein by reference, the disclosure of this application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An article comprising:
a substrate comprising:
a detection zone comprising immobilized antibody that specifically binds Δ-9-tetrahydrocannabinol; and
a fluid collection reservoir in fluid communication with the detection zone.

2. The article of claim 1 wherein the immobilized antibody is immobilized to a porous matrix.

3. The article of claim 1 wherein the detection zone further comprises a control capture area that comprises immobilized antibody that specifically binds a saliva component.

4. (canceled)

5. The article of claim 1 wherein the substrate further comprises a rupturable reservoir comprising a regent solution, wherein when the reservoir is ruptured, the reagent solution is in fluid communication with detection zone.

6. The article of claim 5 wherein the reagent solution comprises a reagent for generating a detectable signal.

7. The article of claim 5 wherein the reagent solution comprises a surfactant solution.

8. The article of claim 1 further comprising a cover that is at least partially transparent and covers at least a portion of the detection zone.

9. The article of claim 1 wherein the substrate comprises a structure of microfluidic transport architecture.

10. A method for detecting Δ-9-tetrahydrocannabinol in a saliva sample, the method comprising:
obtaining a saliva sample from a subject;
contacting at least a portion of the sample with antibody that specifically binds THC; and
detecting THC captured by the antibody.

11. The method of claim 10 wherein the sample is collected within two hours of the subject's most recent use of or exposure to cannabis.

12. The method of claim 10 wherein the method provides a result in no more than seven minutes.

13. The method of claim 10 wherein the method generates a detectable signal at a threshold THC concentration in the saliva sample of 14 ng/mL.

14. An article comprising:
a detection zone comprising an immobilized target compound;
a sample entry zone; and
a reagent zone in fluid communication with the detection zone and the sample entry zone, wherein the reagent zone comprises a ligand that comprises:
specific binding affinity for the immobilized target compound; and
a detectable label.

15. The article of claim 14 wherein the target comprises Δ-9-tetrahydrocannabinol.

16. The article of claim 14 wherein the detection zone comprises the target compound in a complex with a carrier.

17. (canceled)

18. The article of claim 14 further comprising an absorbent reservoir in fluid communication with the detection zone and positioned opposite the reagent zone with respect to the detection zone.

19. The article of claim 14 wherein the detection zone comprises a fluid transport membrane.

20. The article of claim 14 wherein at least one surface providing fluid communication between two zones comprises microfluidic architecture.

21. The article of claim 14 wherein:
the detection zone further comprises a control target; and
the reagent zone comprises a control ligand that comprises:
specific binding affinity for the control target; and
a detectable label.

22. A method for detecting Δ-9-tetrahydrocannabinol in a saliva sample, the method comprising:
obtaining a saliva sample from a subject;
contacting at least a portion of the sample with the article of claim 14; and
detecting a detectable signal generated by the detectable label.

23. The method of claim 22 wherein the sample is collected within two hours of the subject's most recent use of or exposure to cannabis.

24. The method of claim 22 wherein the method provides a result in no more than seven minutes.

25. The method of claim 22 wherein the method generates a detectable signal at a threshold THC concentration in the saliva sample of 14 ng/mL.
